(19) United States
(12) Patent Application Publication
(10) Pub. No.: US 2025/0221753 A1
(43) Pub. Date: Jul. 10, 2025

(54) SYSTEMS AND METHODS FOR PULSED FIELD ABLATION

(71) Applicant: St. Jude Medical, Cardiology Division, Inc., St. Paul, MN (US)

(72) Inventors: Hugo Andres Belalcazar, Minneapolis, MN (US); Derek C. Sutermeister, Ham Lake, MN (US); Isaac Remer, Richfield, MN (US); John Tranter, Minneapolis, MN (US); Lakshya Mittal, Dayton, MN (US)

(21) Appl. No.: 19/013,666

(22) Filed: Jan. 8, 2025

Related U.S. Application Data
(60) Provisional application No. 63/619,548, filed on Jan. 10, 2024.

(51) Int. Cl. A61B 18/00

(52) U.S. Cl. CPC A61B 18/00; A61B 2018/0016; A61B 2018/00613; A61B 2018/0075

(57) ABSTRACT

An electroporation system is provided. The electroporation system includes a catheter including at least one therapeutic electrode, a plurality of return electrodes, a pulse generator configured to apply energy between the at least one therapeutic electrode and the plurality of return electrodes to generate a lesion at the at least one therapeutic electrode, and at least one impedance load, each of the at least one impedance load coupled between the pulse generator and one of the plurality of return electrodes.

### CROSS REFERENCE TO RELATED APPLICATIONS

This application claims priority to U.S. Provisional Patent Application No. 63/619,548 filed on Jan. 10, 2024, which is incorporated by reference herein in its entirety.

### FIELD OF THE DISCLOSURE

The present disclosure relates generally to tissue ablation systems. In particular, the present disclosure relates to improved systems and methods for pulsed field ablation.

### BACKGROUND

It is generally known that ablation therapy may be used to treat various conditions afflicting the human anatomy. For example, ablation therapy may be used in the treatment of atrial arrhythmias. When tissue is ablated, or at least subjected to ablative energy generated by an ablation generator and delivered by an ablation catheter, lesions form in the tissue. Electrodes mounted on or in ablation catheters are used to cause tissue destruction in cardiac tissue to correct conditions such as ventricular and atrial arrhythmias (including, but not limited to, ectopic atrial tachycardia, atrial fibrillation, and atrial flutter).

Arrhythmia (i.e., irregular heart rhythm) can create a variety of dangerous conditions including loss of synchronous atrioventricular contractions and stasis of blood flow which can lead to a variety of ailments and even death. It is believed that the primary cause of atrial arrhythmia is stray electrical signals within the left or right atrium of the heart. The ablation catheter imparts ablative energy (e.g., radiofrequency energy, cryoablation, lasers, chemicals, high-intensity focused ultrasound, etc.) to cardiac tissue to create a lesion in the cardiac tissue. This lesion disrupts undesirable electrical pathways and thereby limits or prevents stray electrical signals that lead to arrhythmias.

Electroporation is a substantially non-thermal ablation technique that involves applying strong electric-fields that induce pore formation in the cellular membrane. The electric field may be induced by applying a relatively short duration pulse which may last, for instance, from a nanosecond to several milliseconds and generate a moderate amount of heating. Such a pulse may be repeated to form a pulse train. When such an electric field is applied to tissue in an in vivo setting, the cells in the tissue are subjected to an increased trans-membrane potential, which opens the pores on the cell plasma membrane. Electroporation may be reversible (i.e., the temporally-opened pores will reseal) or irreversible (i.e., the pores will remain open). For example, in the field of gene therapy, reversible electroporation (i.e., temporarily open pores) is used to transfect high molecular weight therapeutic vectors into the cells. In other therapeutic applications, a suitably configured pulse train alone may be used to cause cell destruction, for instance by causing irreversible electroporation.

For example, pulsed field ablation (PFA) may be used to perform instantaneous pulmonary vein isolation (PVI). PFA generally involves delivering high voltage pulses from electrodes disposed on a catheter. For example, voltage pulses may range from less than about 50 volts to about 10,000 volts or higher. These fields may be applied between pairs of electrodes (bipolar therapy) or between one or more electrodes and a return patch (monopolar therapy).

In PFA, different waveforms may be used to achieve different goals. For example, some waveforms may result in larger or smaller lesion size than other waveforms. Further, some waveforms result in higher or lower overall energy delivery than other waveforms (less overall energy delivery generally corresponds to less heating of the target tissue). As another example, some waveforms are more likely to induce muscular contractions in a patient. Generally, it is desirable to deliver electroporation therapy with a relatively low number of therapy applications over a relatively short timeframe. Further, it is generally desirable to minimize thermal heating of the tissue, and to have little to no skeletal muscle recruitment (i.e., avoiding muscle contractions). In addition, it is also generally desirable to reduce the likelihood of waveforms generating sustained atrial arrhythmias.

### BRIEF SUMMARY OF THE DISCLOSURE

In one aspect, an electroporation system is provided. The electroporation system includes a catheter including at least one therapeutic electrode, a plurality of return electrodes, a pulse generator configured to apply energy between the at least one therapeutic electrode and the plurality of return electrodes to generate a lesion at the at least one therapeutic electrode, and at least one impedance load, each of the at least one impedance load coupled between the pulse generator and one of the plurality of return electrodes.

In another aspect, a method for electroporation therapy is provided. The method includes applying, using a pulse generator, energy between at least one therapeutic electrode on a catheter and a plurality of return electrodes to generate a lesion at the at least one therapeutic electrode, wherein at least one impedance load is coupled between at least one of the plurality of return electrodes and the pulse generator.

The foregoing and other aspects, features, details, utilities, and advantages of the present disclosure will be apparent from reading the following description and claims, and from reviewing the accompanying drawings.

### BRIEF DESCRIPTION OF THE DRAWINGS

FIG. 1 is a schematic and block diagram view of a system for electroporation therapy.

FIGS. 2A and 2B are views of one embodiment of a catheter assembly that may be used with the system shown in FIG. 1.

FIGS. 3A-3C are views of alternative embodiments of a catheter assembly that may be used with the system shown in FIG. 1.

FIG. 4 is a simplified schematic diagram of an example ablation system.

FIG. 5 is one embodiment of a waveform that may be delivered using the system shown in FIG. 1.

### DETAILED DESCRIPTION OF THE DISCLOSURE

The present disclosure provides systems and methods for electroporation. An electroporation system includes a catheter including at least one therapeutic electrode, a plurality of return electrodes, a pulse generator configured to apply energy between the at least one therapeutic electrode and the plurality of return electrodes to generate a lesion at the at least one therapeutic electrode, and at least one impedance load, each of the at least one impedance load coupled between the pulse generator and one of the plurality of return electrodes.

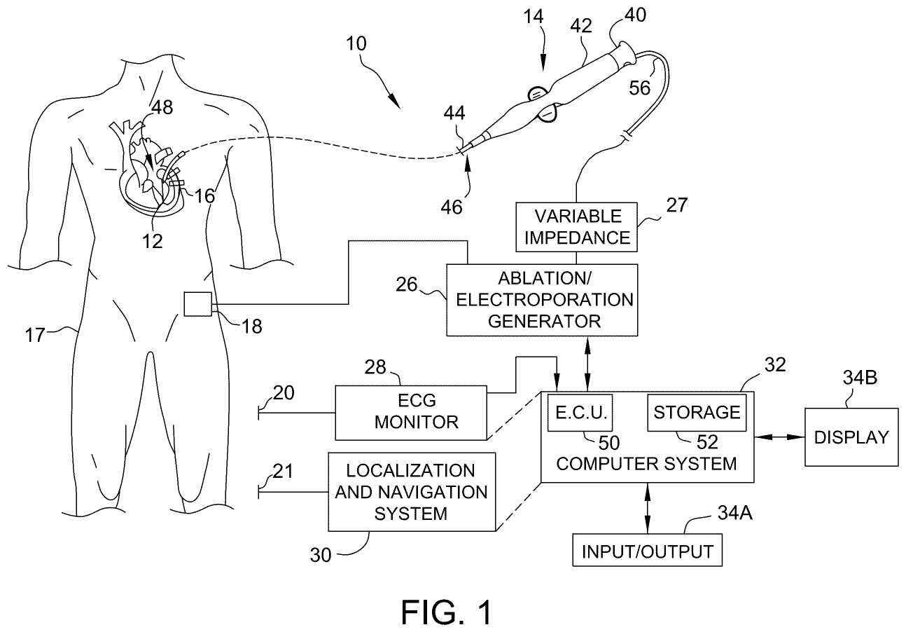

FIG. 1 is a schematic and block diagram view of a system 10 for electroporation therapy. In general, system 10 includes a catheter electrode assembly 12 disposed at a distal end 48 of a catheter 14. As used herein, “proximal” refers to a direction toward the end of the catheter near the clinician and “distal” refers to a direction away from the clinician and (generally) inside the body of a patient. The electrode assembly includes one or more individual, electrically-isolated electrode elements. Each electrode element, also referred to herein as a catheter electrode, is individually wired such that it can be selectively paired or combined with any other electrode element to act as a bipolar or a multi-polar electrode.

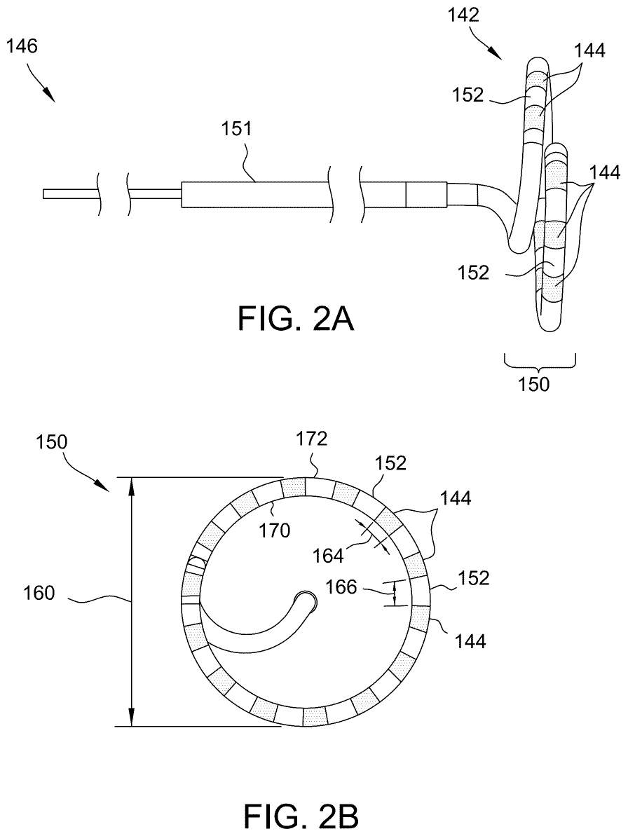

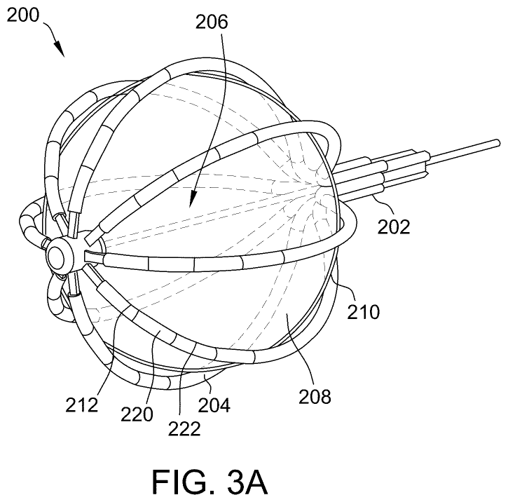

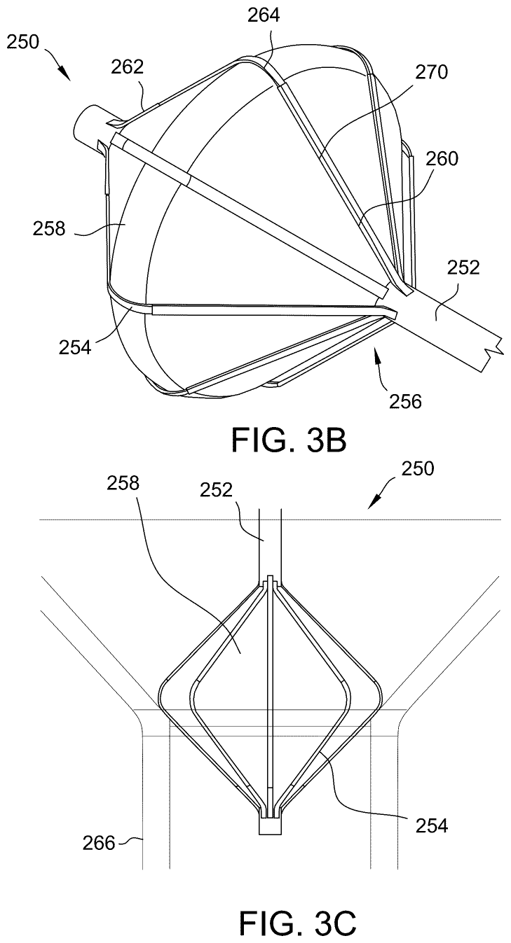

System 10 may be used for irreversible electroporation (IRE) to destroy tissue. In particular, system 10 may be used for electroporation-induced therapy that includes delivering electrical pulses in such a manner as to directly cause an irreversible loss of plasma membrane integrity leading to its breakdown and cell destruction. This mechanism of cell destruction may be viewed as an “outside-in” process, meaning that the disruption of the outside plasma membrane of the cell causes detrimental effects to the inside of the cell. Sometimes these electrical pulses may directly manipulate and damage the intracellular organelles to induce cell death, without causing a significant amount of damage to the cell membrane. Typically, for classical plasma membrane electroporation, electric energy may be delivered as a pulsed electric field in the form of short-duration pulses (e.g., having a 10 nanosecond (ns) to 100 millisecond (ms) duration) between closely spaced electrodes capable of delivering an electric field strength of about 0.05 to 100.0 kilovolts/centimeter (kV/cm). System 10 may be used for high output (e.g., high voltage and/or high current) electroporation procedures. Further, system 10 may be used with a loop catheter such as that depicted in FIGS. 2A and 2B, and/or with a basket catheter such as those depicted in FIGS. 3A-3C. In some embodiments, system 10 is used for reversible electroporation instead of or in addition to irreversible electroporation.

In one embodiment, stimulation is delivered selectively (e.g., between pairs of electrodes) on catheter 14. Further, the electrodes on catheter 14 may be switchable between being connected to a 3D mapping system and being connected to an electroporation generator.

Irreversible electroporation through a multi-electrode catheter may enable pulmonary vein isolation in as few as one shock per vein, which may produce much shorter procedure times compared to sequentially positioning a radiofrequency (RF) ablation tip around a vein. Further, irreversible electroporation may be used for focal ablation procedures. Notably, the embodiments described herein may be used with any suitable irreversible electroporation application.

It should be understood that while the energization strategies are described as involving square wave pulses, embodiments may use variations and remain within the spirit and scope of the disclosure. For example, exponentially-decaying pulses, exponentially-increasing pulses, and combinations may be used.

Further, it should be understood that the mechanism of cell destruction in electroporation is not primarily due to heating effects, but rather to cell membrane disruption (e.g., through pore formation and/or other cell damage) through application of a high-voltage electric field. Thus, electroporation may avoid some possible thermal effects that may occur when using radio frequency (RF) energy. This “colder therapy” thus has desirable characteristics.

With this background, and now referring again to FIG. 1, system 10 includes a catheter electrode assembly 12 including at least one catheter electrode. Electrode assembly 12 is incorporated as part of a medical device such as a catheter 14 for electroporation therapy of tissue 16 in a body 17 of a patient. In the illustrative embodiment, tissue 16 includes heart or cardiac tissue. It should be understood, however, that embodiments may be used to conduct electroporation therapy with respect to a variety of other body tissues (e.g., renal tissue, tumors, etc.).

FIG. 1 further shows a plurality of return electrodes designated 18, 20, and 21, which are diagrammatic of the body connections that may be used by the various sub-systems included in overall system 10, such as an electroporation generator 26, an electrophysiology (EP) monitor such as an ECG monitor 28, and a localization and navigation system 30 for visualization, mapping, and navigation of internal body structures. In the illustrated embodiment, return electrodes 18, 20, and 21 are patch electrodes. It should be understood that the illustration of a single patch electrode is diagrammatic only (for clarity) and that such sub-systems to which these patch electrodes are connected may, and typically will, include more than one patch (body surface) electrode, and may include split patch electrodes (as described herein). Further, in some embodiments, in a multiplexing arrangement, therapy may repeatedly switch between using a different return electrodes 18, 20, 21. In other embodiments, return electrodes 18, 20, and 21 may be any other type of electrode suitable for use as a return electrode including, for example, one or more catheter electrodes. Return electrodes that are catheter electrodes may be part of electrode assembly 12 or part of a separate catheter or device (not shown). System 10 may further include a main computer system 32 (including an electronic control unit 50 and data storage-memory 52), which may be integrated with localization and navigation system 30 in certain embodiments. System 32 may further include conventional interface components, such as various user input/output mechanisms 34A and a display 34B, among other components.

Electroporation generator 26 is configured to energize the electrode element(s) in accordance with an electroporation energization strategy, which may be predetermined or may be user-selectable. For electroporation therapy, generator 26 may be configured to produce an electric energy that is delivered via electrode assembly 12 as a pulsed electric field in the form of short-duration square wave pulses (e.g., a nanosecond to several milliseconds duration, or any duration suitable for electroporation) between closely spaced electrodes capable of delivering an electric field strength (i.e., at the tissue site) of about 0.05 to 100.0 kV/cm. The amplitude and pulse width needed for irreversible electroporation are inversely related. That is, as pulse widths are decreased, the amplitude may generally be increased to achieve chronaxie. The electric energy may be delivered, for example, using a fixed voltage delivery system (in which a fixed voltage is applied, independent of a patient impedance) or using a fixed current delivery system (in which a fixed current is achieved by adjusting the voltage based on the patient impedance). In a fixed current delivery system, the patient impedance may be determined, for example, by delivering a relatively small voltage pulse and measuring current to calculate impedance, or by delivering an AC current waveform and measuring voltage to calculate impedance. A fixed current system may also involve measuring current (e.g., before or during therapy delivery) and adjusting voltage accordingly. For example, current may be measured during delivery of a first therapy pulse (or during a pre-therapy pulse with a relatively low voltage), an impedance may be calculated from the measured current, and the voltage may be adjusted (and then left unchanged) to obtain the desired current during therapy. In another example, current may be measured during one or more pulses delivered during therapy, the impedance may be calculated for each pulse that the current was measured for, and the voltage of each subsequent pulse may be actively adjusted.

Electroporation generator 26, sometimes also referred to herein as a DC energy source, is a biphasic electroporation generator 26 configured to generate a series of energy pulses that all produce current in two directions (i.e., positive and negative pulses). In other embodiments, electroporation generator is a monophasic or polyphasic electroporation generator. In some embodiments, electroporation generator 26 is configured to output energy in pulses at selectable energy levels, such as fifty joules, one hundred joules, two hundred joules, and the like. Other embodiments may have more, or fewer energy settings and the values of the available setting may be the same or different (settings may include, e.g., waveform parameters, voltage, current, number of applications, etc.). For successful electroporation, some embodiments utilize the two hundred joule output level. For example, electroporation generator 26 may output a pulse having a peak magnitude from about 10 Volts (V) to about 20,000 V. Other embodiments may output any other suitable positive or negative voltage.

In some embodiments, a variable impedance 27 allows the impedance of system 10 to be varied to limit arcing. Moreover, variable impedance 27 may be used to change one or more characteristics, such as amplitude, duration, pulse shape, and the like, of an output of electroporation generator 26. Although illustrated as a separate component, variable impedance 27 may be incorporated in catheter 14 or generator 26.

With continued reference to FIG. 1, as noted above, catheter 14 may include functionality for electroporation and in certain embodiments also additional ablation functions (e.g., RF ablation). It should be understood, however, that in those embodiments, variations are possible as to the type of ablation energy provided (e.g., cryoablation, ultrasound, etc.).

In the illustrative embodiment, catheter 14 includes a cable connector or interface 40, a handle 42, and a shaft 44 having a proximal end 46 and a distal 48 end. Catheter 14 may also include other conventional components not illustrated herein such as a temperature sensor, additional electrodes, and corresponding conductors or leads. Connector 40 provides mechanical and electrical connection(s) for cable 56 extending from generator 26. Connector 40 may include conventional components known in the art and as shown is disposed at the proximal end of catheter 14.

Handle 42 provides a location for the clinician to hold catheter 14 and may further provide means for steering or the guiding shaft 44 within body 17. For example, handle 42 may include means to change the length of a guidewire extending through catheter 14 to distal end 48 of shaft 44 or means to steer shaft 44. Moreover, in some embodiments, handle 42 may be configured to vary the shape, size, and/or orientation of a portion of the catheter, and it will be understood that the construction of handle 42 may vary. In an alternate embodiment, catheter 14 may be robotically driven or controlled. Accordingly, rather than a clinician manipulating a handle to advance/retract and/or steer or guide catheter 14 (and shaft 44 thereof in particular), a robot is used to manipulate catheter 14. Shaft 44 is an elongated, tubular, flexible member configured for movement within body 17. Shaft 44 is configured to support electrode assembly 12 as well as contain associated conductors, and possibly additional electronics used for signal processing or conditioning. Shaft 44 may also permit transport, delivery and/or removal of fluids (including irrigation fluids and bodily fluids), medicines, biologics, and/or surgical tools or instruments. Shaft 44 may be made from conventional materials such as polyurethane and defines one or more lumens configured to house and/or transport electrical conductors, fluids or surgical tools, as described herein. Shaft 44 may be introduced into a blood vessel or other structure within body 17 through a conventional introducer. Shaft 44 may then be advanced/retracted and/or steered or guided through body 17 to a desired location such as the site of tissue 16, including through the use of guidewires or other means known in the art.

Localization and navigation system 30 may be provided for visualization, mapping and navigation of internal body structures. Localization and navigation system 30 may include conventional apparatus known generally in the art. For example, localization and navigation system 30 may be substantially similar to the EnSite Precision™ System, commercially available from Abbott Laboratories, and as generally shown in commonly assigned U.S. Pat. No. 7,263,397 titled “Method and Apparatus for Catheter Navigation and Location and Mapping in the Heart”, the entire disclosure of which is incorporated herein by reference. In another example, localization and navigation system 30 may be substantially similar to the EnSite X™ System, as generally shown in U.S. Pat. App. Pub. No. 2020/0138334 titled “Method for Medical Device Localization Based on Magnetic and Impedance Sensors”, the entire disclosure of which is incorporated herein by reference. It should be understood, however, that localization and navigation system 30 is an example only, and is not limiting in nature. Other technologies for locating/navigating a catheter in space (and for visualization) are known, including for example, the CARTO navigation and location system of Biosense Webster, Inc., the Rhythmia® system of Boston Scientific Scimed, Inc., the KODEX® system of Koninklijke Philips N.V., the AURORA® system of Northern Digital Inc., commonly available fluoroscopy systems, or a magnetic location system such as the gMPS system from Mediguide Ltd.

In this regard, some of the localization, navigation and/or visualization systems may include a sensor for producing signals indicative of catheter location information, and may include, for example, one or more electrodes in the case of an impedance-based localization system, or alternatively, one or more coils (i.e., wire windings) configured to detect one or more characteristics of a magnetic field, for example in the case of a magnetic-field based localization system. As yet another example, system 10 may utilize a combination electric field-based and magnetic field-based system as generally shown with reference to U.S. Pat. No. 7,536,218 entitled “Hybrid Magnetic-Based and Impedance Based Position Sensing,” the disclosure of which is incorporated herein by reference in its entirety.

Pulsed field ablation (PFA), which is a methodology for achieving irreversible electroporation and cell death, may be implemented using the systems and methods described herein. In some cases, PFA may be used at specific cardiac tissue sites such as the pulmonary veins to perform a pulmonary vein isolation (PVI), or to perform focal ablation. In PFA, electric fields may be applied between adjacent electrodes (in a bipolar approach) or between one or more electrodes and a return patch (in a monopolar approach). There are advantages and disadvantages to each of these approaches.

Both approaches, using an appropriate electrode geometry, are able to provide contiguous lesions. For lesion size and proximity, the monopolar approach can potentially create deeper lesions with the same applied voltage. Further, the monopolar approach may be able to create lesions from a distance (e.g., generally proximate, but not necessarily contacting tissue). The bipolar approach may create smaller lesions, requiring closer proximity or contact with tissue to create transmural lesions (depending on, for example, tissue thickness). To monitor operation of system 10, one or more impedances between catheter electrodes 144 (also referred to herein as therapeutic electrodes) and/or return electrodes 18, 20, and 21 may be measured. For example, for system 10, impedances may be measured as described in U.S. Patent Application Publication No. 2019/0117113, filed on Oct. 23, 2018, U.S. Patent Application Publication No. 2019/0183378, filed on Dec. 19, 2018, and U.S. Patent Application No. 63/027,660, filed on May 20, 2020, all of which are incorporated by reference herein in their entirety.

FIGS. 2A and 2B are views of one embodiment of a catheter assembly 146 that may be used with catheter 14 in system 10. Catheter assembly 146 may be referred to as a loop catheter.

Those of skill in the art will appreciate that, in other embodiments, any suitable catheter may be used. That is, the systems and methods described herein are not limited to use with the particular catheter assemblies shown. For example, the systems and methods described herein may be implemented in a linear catheter, a grid catheter (e.g., a catheter including a number of splines arranged in a plane, each spline including one or more electrodes), a paddle-shaped catheter, a disc-shaped catheter, and/or a focal ablation catheter (e.g., such as the Abbott TactiFlex catheter and TactiCath catheter).

Specifically, FIG. 2A is a side view of catheter assembly 146 with a variable diameter loop 150 at a distal end 142. FIG. 2B is an end view of variable diameter loop 150 of catheter assembly 146. Those of skill in the art will appreciate that the methods and systems described herein may be implemented using any suitable catheter (e.g., fixed loop catheters, linear catheters, multi-spline catheters, disc catheters, basket catheters, etc.). As shown in FIGS. 2A and 2B, variable diameter loop 150 is coupled to a distal section 151 of shaft 44.

Variable diameter loop 150 is selectively transitionable between an expanded (also referred to as “open”) diameter 160 (shown in FIG. 2A) and a retracted (also referred to as “closed”) diameter 160 (not shown). In the example embodiment, an expanded diameter 160 is twenty eight mm and a retracted diameter 160 is fifteen mm. In other embodiments, diameter 160 may be variable between any suitable open and closed diameters 160.

In the embodiment shown, variable diameter loop 150 includes fourteen catheter electrodes 144 substantially evenly spaced around the circumference of variable diameter loop 150 in the expanded configuration. Catheter electrodes 144 may also be referred to as therapeutic electrodes. In the retracted configuration, one or more of electrodes 144 may overlap. In other embodiments, other arrangements of catheter electrodes 144 may be implemented. For example, in one embodiment, variable diameter loop 150 includes twelve catheter electrodes 144.

Catheter electrodes 144 are platinum ring electrodes configured to conduct and/or discharge electrical current in the range of one thousand volts and/or ten amperes. In other embodiments, variable diameter loop 150 may include any suitable number of catheter electrodes 144 made of any suitable material. Catheter electrodes 144 may include any catheter electrode suitable to conduct high voltage and/or high current (e.g., in the range of one thousand volts and/or ten amperes). Each catheter electrode 144 is separated from each other catheter electrode by an insulated gap 152. In the example embodiment, each catheter electrode 144 has a same length 164 (shown in FIG. 2B) and each insulated gap 152 has a same length 166 as each other gap 152. Length 164 and length 166 are both about 2.5 mm in the example embodiment. In other embodiments, length 164 and length 166 may be different from each other. Moreover, in some embodiments, catheter electrodes 144 may not all have the same length 164 and/or insulated gaps 152 may not all have the same length 166. In some embodiments, catheter electrodes 144 are not spaced evenly around the circumference of variable diameter loop 150.

Diameter 160 and catheter electrode 144 spacing may be developed to provide a targeted range of energy density to tissue, as well as to provide sufficient electroporation coverage for different human anatomic geometries. In general, a sufficient number of electrodes 144 with appropriate lengths 164 are desired to provide substantially even and continuous coverage around the circumference of variable diameter loop 150, while still allowing enough flexibility to allow variable diameter loop 150 to expand and contract to vary diameter 160 to the desired extremes.

As mentioned above, length 164 of catheter electrodes 144 may be varied. Increasing length 164 of catheter electrodes 144 may increase coverage of electrodes 144 around the circumference of variable diameter loop 150 while also decreasing current density (by increasing the surface area) on electrodes 144, which may help prevent arcing and reduce thermal effects during electroporation operations. Increasing length 164 too much, however, may prevent variable diameter loop 150 from forming a smooth circular shape and may limit the closed diameter 160 of variable diameter loop 150. Additionally, too great a length 164 may increase the surface area of catheter electrodes 144 to a point that the current density applied to catheter electrodes 144 by a power source is below the minimum current density needed for successful therapy. Conversely, decreasing length 164 decreases the surface area, thereby increasing the current density (assuming no other system changes) on catheter electrodes 144. As discussed above, greater current densities may lead to increased risk of arcing and heating during electroporation, and may result in larger additional system resistances needing to be added to prevent arcing. Moreover, in order to get a desired, even coverage about the circumference of variable diameter loop 150, more catheter electrodes 144 may be needed if length 164 is decreased. Increasing the number of catheter electrodes 144 on variable diameter loop 150 may prevent variable diameter loop 150 from being able to be contracted to a desired minimum diameter 160.

FIG. 3A is a perspective view of an alternative catheter assembly 200 that may be used with catheter 14. Catheter assembly 200 may be referred to as a basket catheter. Catheter assembly 200 includes a shaft 202 and a plurality of splines 204 surrounding a distal portion 206 of shaft 202. In this embodiment, catheter assembly 200 also includes a balloon 208 enclosed by splines 204. Balloon 208 may be selectively inflated to fill the space between splines 204. Notably, balloon 208 functions as an insulator, and generally reduces energy losses, which may result in increased lesion size. In some embodiments, balloon 208 may be filled with a cold media (e.g., a cryofluid or cold saline). Further, in some embodiments balloon 208 may be a double-layered balloon.

Each spline 204 includes a proximal end 210 coupled to shaft 202 and a distal end 212 coupled to shaft 202. From proximal end 210 to distal end 212, spline 204 has an arcuate shape that extends radially outward.

In this embodiment, each spline 204 includes a plurality of individual electrodes 220. For example, each spline 204 may include an elastic material (e.g., Nitinol) covered in a polymer tube 222, with individual electrodes 220 attached to an exterior of polymer tube 222. In the embodiment shown, each spline 204 includes two electrodes 220. Further, as shown in FIG. 2, electrodes 220 are generally positioned closer to distal end 212 than proximal end 210 to correspond to portions of spline 204 that will contact the pulmonary vein.

Alternatively, each spline 204 may include any suitable number and arrangement of electrodes 220. For example, in some embodiments, each spline 204 includes four electrodes 220.

In this embodiment, alternating splines 204 alternate polarities. That is, electrodes 220 on a particular spline 204 have the same polarity, but electrodes 220 on a particular spline 204 have a different polarity than electrodes 220 on adjacent splines 204. Alternatively, any suitable polarization scheme may be used. During delivery, splines 204 may be collapsed in towards shaft 202. Subsequently, to perform ablation, splines 204 are deployed to extend radially outward.

Splines 204 may all have the same length, or at least some of splines 204 may have different lengths. Further, insulating material on each spline 204 may have the same length, or at least some splines 204 may have insulating material with different lengths. In addition, in some embodiments, catheter assembly 200 includes a distal electrode (not shown) positioned distal of splines 204. The distal electrode may be used to perform point ablation (e.g., by creating a bipole between the distal electrode and one of splines 204), and/or may be used for visualization/mapping purposes (e.g., using the distal electrode in combination with an electrode on shaft 202).

FIG. 3B is a perspective view of an alternative catheter assembly 250 that may be used with catheter 14, and FIG. 3C is a side schematic view of catheter assembly 250. Like catheter assembly 200 (shown in FIG. 3A), catheter assembly 250 may be referred to as a basket assembly.

Catheter assembly 250 includes a shaft 252 and a plurality of splines 254 surrounding a distal portion 256 of shaft 252. In this embodiment, catheter assembly 250 includes a balloon 258 enclosed by splines 254. Balloon 258 may be selectively inflated to occupy the space between splines 254. Notably, balloon 258 functions as an insulator, and generally reduces energy, which may result in increased lesion size.

Each spline 254 includes a proximal end 260 coupled to shaft 252 and a distal end 262 coupled to shaft 252. From proximal end 260, spline 1004 extends radially outward to an inflection point 264, and then extends radially inward to distal end 262. FIG. 3C shows catheter assembly 250 positioned within the pulmonary vein 266.

A body of each spline 254 is made of an elastic material (e.g., Nitinol), and functions as a relatively large electrode. In this embodiment, alternating splines 254 alternate polarities. That is, each positive spline 254 is positioned between two negative splines 254 and vice-versa. Alternatively, any suitable polarization scheme may be used.

To control the ablation zone of each spline 254, portions of each spline 254 may be covered with insulating material 270 (e.g., heat-shrink or polymer tubing or spray or dip coat with polyimide or PEBAX), and the exposed portions of splines 254 function as electrodes. In the embodiment shown in FIGS. 3B and 3C, inflection point 264 and portions of spline 254 between inflection point 264 and distal end 262 are generally exposed, while portions of spline 254 between inflection point 264 and proximal end 260 are generally insulated. This results in the portions of spline 254 that contact pulmonary vein 266 being exposed (see FIG. 3C). Alternatively, any suitable insulation configuration may be used.

During delivery, splines 254 and balloon 258 may be collapsed. To perform ablation, splines 254 are deployed with inflection points 264 extending radially outward, and balloon 258 is selectively inflated to occupy the space between splines 254.

The combination of balloon 258 and splines 254 facilitates straightforward delivery and deployment of catheter assembly 250. Further, balloon 258 drives more energy into ablated tissue, and stabilizes splines 254 to prevent lateral movement. In addition, using splines 254 as electrodes instead of individual smaller electrodes may facilitate reducing the cost and increasing the reliability of catheter assembly 250.

Splines 254 may all have the same length, or at least some of splines 254 may have different lengths. Further, insulating material 270 on each spline 254 may have the same length, or at least some splines 254 may have insulating material 270 with different lengths. In addition, in some embodiments, catheter assembly 250 includes a distal electrode (not shown) positioned distal of splines 254. The distal electrode may be used to perform point ablation (e.g., by creating a bipole between the distal electrode and one of splines 254), and/or may be used for visualization/mapping purposes (e.g., using the distal electrode in combination with an electrode on shaft 252).

Those of skill the art will appreciate that catheter assembly 146 (shown in FIGS. 2A and 2B), catheter assembly 200 (shown in FIG. 3A), and catheter assembly 250 (shown in FIGS. 3B and 3C) are merely examples. Notably, the systems and methods described herein may be implemented using any suitable catheter assembly.

For electroporation therapy, waveforms are generated using a pulse generator (e.g., electroporation generator 26 (shown in FIG. 1)) and applied between two or more catheter electrodes (i.e., a bipolar approach) and/or between individual catheter electrodes and a return patch (i.e., a monopolar approach). The waveforms may be monophasic, biphasic (i.e., having both a positive pulse and a negative pulse), or polyphasic. Further, the waveforms may include one or more bursts of pulses (with each burst including multiple pulses). Further, the waveforms are defined by multiple parameters (e.g., pulse width, pulse amplitude, frequency, etc.).

Different waveforms may be used to achieve different goals. For example, some waveforms may result in larger or smaller lesion size than other waveforms. Further, some waveforms result or higher or lower overall energy delivery than other waveforms (less overall energy delivery generally corresponds to less heating of the target tissue). As another example, some waveforms are more likely to induce muscular contractions in a patient. Generally, it is desirable to deliver electroporation therapy with a relatively low number of therapy applications over a relatively short timeframe. Further, it is generally desirable to avoid thermal heating of the tissue, and to have little to no skeletal muscle recruitment (i.e., avoiding muscle contractions).

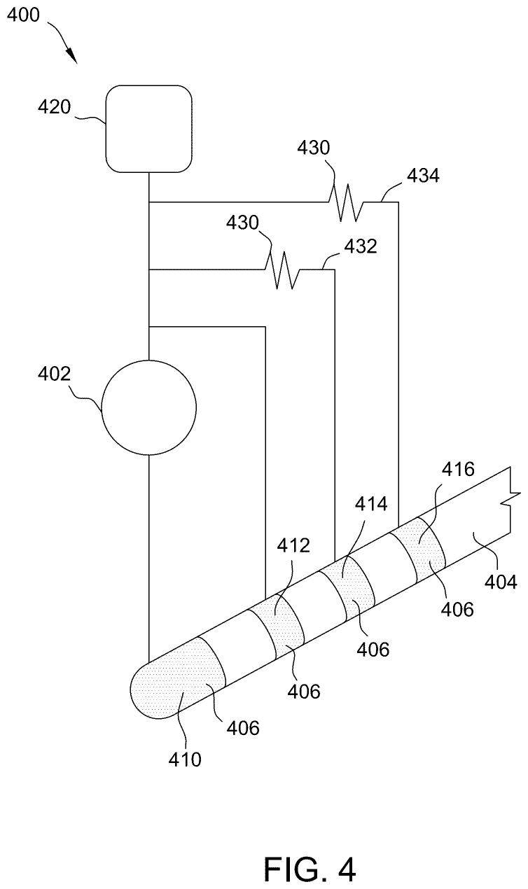

FIG. 4 is a simplified schematic diagram of an example ablation system 400, such as system 10 (shown in FIG. 1). Ablation system 400 includes a pulse generator 402 (such as electroporation generator 26 (shown in FIG. 1)) and a catheter 404 (such as catheter 14 (shown in FIG. 1)). In this embodiment, catheter 404 is a linear catheter that includes four electrodes 406. Specifically, electrodes 406 include a tip electrode 410, a distal ring electrode 412, an intermediate ring electrode 414, and a proximal ring electrode 416. Notably, catheter 404 is merely an example, and any suitable catheter design (e.g., those shown in FIGS. 2A-3C) including any suitable number and arrangement of electrodes may be used.

In at least some known ablation systems, for bipolar therapy, energy is delivered between two or more electrodes in close proximity to one another on a catheter (e.g., for catheter 404, energy may be delivered between tip electrode 410 and at least one of distal ring electrode 412, intermediate ring electrode 414, and proximal ring electrode 416). In contrast, for monopolar therapy, in at least some known ablation systems, energy is delivered between at least one electrode on the catheter and an external patch electrode (e.g., return electrodes 18, 20, and 21 (shown in FIG. 1)). As noted above, monopolar and bipolar approaches each have associated advantages and disadvantages.

In contrast to at least some known ablation systems, the embodiments described herein combine monopolar and bipolar approaches, delivering energy to some or all available electrodes, and limiting energy to certain paths using one or more impedance loads (e.g., resistive loads, capacitive loads, and/or inductive loads), as described herein. This allows an intended therapy electrode (i.e., the electrode at which the lesion is to be generated) to receive relatively high current, while maintaining lower energy levels at other regions where lesion generation is not desirable. Further, when delivering energy to one or more external patch electrodes, the impedance load(s) may be used to reduce or eliminate skeletal muscle recruitment (SMR), produce equivalent lesion depth with less power than at least some known systems (or greater lesion depth with the same power as at least some known systems), and produce less lesion width (i.e., less lesion shadow). This is because the lesion will be generated primarily at the lesion generating electrode, and will not be generated as an elongated lesion between a pair of electrodes.

Those of skill in the art will appreciate that all circuits inherently have an associated impedance (e.g., even a conductive wire having a non-zero length will have some non-zero level of impedance). Notably, as used herein, an “impedance load” refers to an additional impedance load beyond those inherent in existing circuitry.

Referring back to FIG. 4, in this example, ablation system 400 further includes an external patch electrode 420. Although only one external patch electrode 420 is shown, some embodiments may include multiple external patch electrodes 420. External patch electrode 420, as well as electrodes 406, are each electrically coupled to pulse generator 402. Ablation system 400 also includes one or more impedance loads 430. Specifically, in the example shown in FIG. 4, ablation system 400 includes a first resistive load 432 coupled between pulse generator 402 and intermediate ring electrode 414, and a second resistive load 434 coupled between pulse generator 402 and proximal ring electrode 416. In one example implementation, first resistive load 432 has a resistance of approximately 53 Ohms, and second resistive load 434 has a resistance of approximately 61 Ohms. Notably, the resistance values are merely an example, and ablation system 400 may include any suitable number and configuration of impedance loads 430. For example, in some embodiments, first resistive load 432 may have a resistance in a range from 30-70 Ohms, and second resistive load 434 may have a resistance in a range from 40-80 Ohms.

Although impedance loads 430 are resistive loads 430 and 432 in this example, in other embodiments, impedance loads 430 may additionally or alternatively include inductive loads and/or capacitive loads (i.e., such that impedance loads 430 constitute resistive, inductive, and/or capacitive loads). The inductive and/or capacitive loads may introduce a phase shift between electrodes, which facilitates controlling lesion formation and directing the electric field.

In the example shown in FIG. 4, the full voltage of pulses generated by pulse generator 402 is applied between tip electrode 410 and distal ring electrode 412, and between tip electrode 410 and external patch electrode 420. However, due to first and second resistive loads 432 and 434, a reduced voltage is applied between tip electrode 410 and intermediate ring electrode 414, and between tip electrode 410 and proximal ring electrode 416.

The weighted (i.e., reduced) voltages at intermediate ring electrode 414 and proximal ring electrode 416 shape the electric field into a pear shape (instead of the elongated stadium shape of conventional bipolar approaches), with the widest part of the electric field located near tip electrode 410. By increasing the impedance to intermediate ring electrode 414 and proximal ring electrode 416, lesions proximate to these electrodes may be reduced or completely eliminated, while still realizing relatively high currents at tip electrode 410. Using impedance loads that limit current to intermediate ring electrode 414 and proximal ring electrode 416 to less than 3 amperes (at a pulse amplitude of, for example 2100 V) each may prevent lesion generation at those electrodes.

By providing several paths of current to and from tip electrode 410, the current in intermediate ring electrode 414 and proximal ring electrode 416 is substantially reduced, which facilitates reducing SMR. The current may be further reduced by including another impedance load (e.g., a resistive load) between pulse generator 402 and external patch electrode 420. For example, the impedance load between pulse generator 402 and external patch electrode 420 may limit the current to the external patch electrode 420 to less than 12 amperes, or less than 8 amperes, which facilitates eliminating non-phrenic related muscle recruitment (at a pulse amplitude of, for example 2100 V). Further, it was observed that current levels of 4 amperes or more to external patch electrode 420 still provide substantial lesion depth at tip electrode 410 (at a pulse amplitude of, for example 2100 V).

As used herein, an impedance load coupled between a pulse generator and a return electrode may be implemented as a resistance level incorporated into the return electrode itself. For example, a patch return electrode may have a predetermined resistance level to facilitate limiting current as described herein. This predetermined resistance level would constitute an impedance loud coupled between a pulse generator and the patch return electrode, as the terminology is used herein.

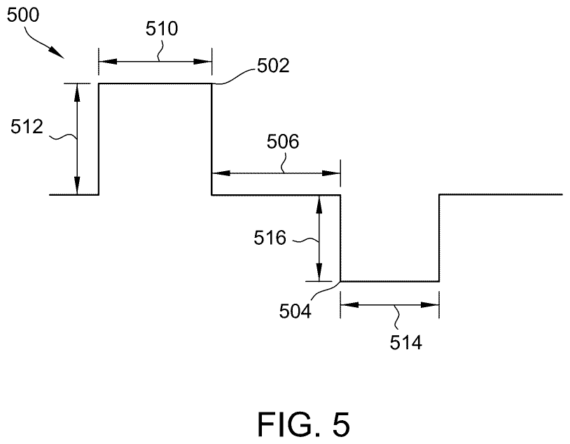

FIG. 5 is one embodiment of a waveform 500 that may be delivered using pulse generator 402 (shown in FIG. 4). Waveform 500 includes a positive pulse 502 followed by a negative pulse 504. Further, there is an intrapulse delay 506 between the positive and negative pulses 502 and 504.

As shown in FIG. 5, positive pulse 502 has a first pulse width 510 and a first pulse amplitude 512. Similarly, negative pulse 504 has a second pulse width 514 and a second pulse amplitude 516. Waveform 500 may be symmetric (i.e., with first pulse width 510 and first pulse amplitude 512 substantially equal to second pulse width 514 and second pulse amplitude 516) or asymmetric (i.e., with at least one of first pulse width 510 and first pulse amplitude 512 different from second pulse width 514 and second pulse amplitude 516).

When first pulse amplitude 512 and second pulse amplitude 516 are both non-zero, waveform 500 is biphasic (i.e., as shown in FIG. 5). For a monophasic waveform, one of first pulse amplitude 512 and second pulse amplitude 516 is zero. For example, if first pulse amplitude 512 is zero, waveform 500 is monophasic with single negative pulse 504. If second pulse amplitude 516 is zero, waveform 500 is monophasic with single positive pulse 502.

In one biphasic example, first and second pulse widths 510 and 514 may each be 3 microseconds (3 μs), with an intrapulse delay of 1 μs. This may be referred to as a 3-1-3 waveform (i.e., first pulse width of 3 μs-intrapulse delay of 1 μs—second pulse width of 3 μs). First and second pulse amplitudes 512 and 516 may each be, for example, on the order of 1800 Volts (1800V).

In one monophasic example, first pulse width 510 is 0 μs, and second pulse width 514 is 3 μs, with an intrapulse delay of 1 μs. This may be referred to as a 0-1-3 waveform (i.e., first pulse width of 0 μs-intrapulse delay of 1 μs-second pulse width of 3 μs). Second pulse amplitude 516 may be, for example, on the order of 1800V. In another example, the intrapulse delay may be 0 μs.

The following Table 1 includes experimental data observed for a 1.52-1-1.52 waveform (i.e., first pulse width of 1.52 μs-intrapulse delay of 1 μs-second pulse width of 1.52 μs) delivered between various electrodes in ablation system 400, with various resistive loads implemented. Notably, these are only example values.

TABLE 1

| | | | Distal Ring | Patch |
|---|---|---|---|---|
| | Average | Tip Average | Average | Average |
| Configuration | Voltage | Current (A) | Current (A) | Current (A) |
| Tip-to-Distal Ring (DR) | 2170.5 | 13.6 | 13.08 | N/A |
| Tip-to-DR & Patch w/150Ω resistor | 2193.5 | 16.8 | 10.8 | 5.6 |
| Tip-to-DR & Patch w/100Ω resistor | 2204 | 17.5 | 10.1 | 6.9 |
| Tip-to-DR & Patch w/47Ω resistor | 2180.5 | 19.5 | 9.6 | 9.7 |
| Tip-to-DR & Patch w/o resistor | 2163.5 | 22.6 | 7.7 | 16.1 |
| Tip-to-Patch | N/A | 18.3 | N/A | 19.3 |

As shown in Table 1, the current at tip electrode 410 may be increased by 25% or more, while maintaining a current at external patch electrode 420 below 10 amperes. This provides substantial benefits to lesion size while maintaining low SMR.

The following Table 2 includes experimental data observed for a 2-1-2 waveform (i.e., first pulse width of 2 μs-intrapulse delay of 1 μs-second pulse width of 2 μs) delivered between various electrodes in ablation system 400, with various resistive loads implemented.

TABLE 2

| | | Tip | Distal Ring | Patch |
|---|---|---|---|---|
| | Average | Average | Average | Average |
| Configuration | Voltage | Current (A) | Current (A) | Current (A) |
| Tip-to-Distal | 2198 | 13.51 | 12.98 | N/A |
| Ring (DR) | | | | |
| Tip-to-DR & Patch | 2163 | 17.78 | 10.28 | 7.11 |
| w/100Ω | | | | |
| Tip-to-DR & Patch | 2157 | 23.23 | 7.90 | 14.63 |
| w/o resistor | | | | |
| Tip-to-Patch | 1967 | 16.48 | N/A | 16.14 |

Of note, including a relatively large resistive load (e.g., 100 Ohms or more) in series with external patch electrode 420 may enable wider pulse widths (e.g., 3-1-3 or greater) while still increasing total current to the therapy electrode (e.g., tip electrode 410). Upper limits on the value for the resistive load generally depend on, for example, pulse widths and voltages of the delivered waveforms. At longer pulse widths and/or higher voltages, the resistance to external patch electrode 420 will generally increase. In general, waveforms that produce acceptable SMR may have deliver approximately 5-30 A of current to external patch electrode 420, with a current threshold changing based on the waveform. Accordingly, the particular waveform and alternate grounding paths will determine the appropriate resistive load in series with external patch electrode 420.

In accordance with the embodiments described herein, it was experimentally verified that, as compared to a monopolar approach, using the same voltage as the monopolar approach, but delivering energy between i) tip electrode 410 and ii) distal ring electrode 412, intermediate ring electrode 414, proximal ring electrode 416, and external patch electrode 420 resulted in deeper lesions. Further, as compared to the monopolar approach, with the same current at tip electrode 410 as the monopolar approach, delivering energy between i) tip electrode 410 and ii) distal ring electrode 412, intermediate ring electrode 414, proximal ring electrode 416, and external patch electrode 420 resulted in lesions of similar depths, but at substantially less voltage and power. Further, current flowing towards the back of the patient was appreciably reduced, and the current to external patch electrode 420 was appreciably reduced (e.g., by 56%), reducing SMR effects.

As noted above, the shape of the electric field may be modified by controlling the voltages delivered to each electrode. In the example of FIG. 4, the difference in the voltage applied at tip electrode 410 and distal ring electrode 412 versus the voltage applied at intermediate ring electrode 414 and proximal ring electrode 416 is responsible for the field shaping. The difference in voltage determines the amount of current, and thus the occurrence of shadow lesion on intermediate ring electrode 414 and proximal ring electrode 416.

Desirable shaping may be achieved, for example, by applying voltages at intermediate ring electrode 414 and proximal ring electrode 416 that are relatively close to the voltage at distal ring electrode 412. For example, if distal ring electrode 412 is at 2100 V, and intermediate ring electrode 414 and proximal ring electrode 416 are at 2000 V, the electric field generated at intermediate ring electrode 414 and proximal ring electrode 416 may push the electric field generated at distal ring electrode 412 towards tip electrode 410. Of course, these voltage values are merely an example, and any suitable values may be used.

The systems and methods described herein are directed to electroporation. An electroporation system includes a catheter including at least one therapeutic electrode, a plurality of return electrodes, a pulse generator configured to apply energy between the at least one therapeutic electrode and the plurality of return electrodes to generate a lesion at the at least one therapeutic electrode, and at least one impedance load, each of the at least one impedance load coupled between the pulse generator and one of the plurality of return electrodes.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. An electroporation system comprising:
a catheter comprising at least one therapeutic electrode;
a plurality of return electrodes;
a pulse generator configured to apply energy between the at least one therapeutic electrode and the plurality of return electrodes to generate a lesion at the at least one therapeutic electrode; and
at least one impedance load, each of the at least one impedance load coupled between the pulse generator and one of the plurality of return electrodes.

2. The electroporation system in accordance with claim 1, wherein the plurality of return electrodes comprises at least one external patch electrode.

3. The electroporation system in accordance with claim 2, wherein the at least one impedance load comprises an impedance load coupled between the pulse generator and the at least one external patch electrode.

4. The electroporation system in accordance with claim 2, wherein the at least one external patch electrode comprises a plurality of external patch electrodes.

5. The electroporation system in accordance with claim 1, wherein at least some of the plurality of return electrodes are located on the same catheter as the at least one therapeutic electrode.

6. The electroporation system in accordance with claim 1, wherein the at least one impedance load comprises at least one resistive load, at least one inductive load, and/or at least one capacitive load.

7. The electroporation system in accordance with claim 1, wherein the at least one pulse generator is configured to apply a monophasic and/or biphasic pulse between the at least one therapeutic electrode and the plurality of return electrodes.

8. The electroporation system in accordance with claim 1, wherein the at least one therapeutic electrode comprises a tip electrode on the catheter.

9. The electroporation system in accordance with claim 1, wherein the catheter comprises a basket catheter.

10. The electroporation system in accordance with claim 1, wherein the catheter comprises a linear catheter.

11. The electroporation system in accordance with claim 1, wherein the catheter comprises a loop catheter, a grid catheter, a paddle-shaped catheter, and/or a disc-shaped catheter.

12. A method for electroporation therapy, the method comprising:
applying, using a pulse generator, energy between at least one therapeutic electrode on a catheter and a plurality of return electrodes to generate a lesion at the at least one therapeutic electrode, wherein at least one impedance load is coupled between at least one of the plurality of return electrodes and the pulse generator.

13. The method in accordance with claim 12, wherein the plurality of return electrodes includes at least one external patch electrode.

14. The method in accordance with claim 13, wherein the at least one impedance load includes an impedance load coupled between the pulse generator and the at least one external patch electrode.

15. The method in accordance with claim 13, wherein the at least one external patch electrode includes a plurality of external patch electrodes.

16. The method in accordance with claim 12, wherein at least some of the plurality of return electrodes are located on the same catheter as the at least one therapeutic electrode.

17. The method in accordance with claim 12, wherein the at least one impedance load includes at least one resistive load, at least one inductive load, and/or at least one capacitive load.

18. The method in accordance with claim 12, wherein applying energy comprises applying a monophasic and/or biphasic pulse between the at least one therapeutic electrode and the plurality of return electrodes.

19. The method in accordance with claim 12, wherein the plurality of return electrodes have varying associated impedances to maintain a similar current density across the plurality of return electrodes.

20. The method in accordance with claim 12, wherein the catheter is at least one of a loop catheter, a linear catheter, a multi-spline catheter, a disc-shaped catheter, a basket catheter, and a grid catheter.
